(19) United States
(12) Patent Application Publication
(10) Pub. No.: US 2024/0318109 A1
(43) Pub. Date: Sep. 26, 2024

(54) CONSUMABLE FOR A BIOREACTOR SYSTEM

(71) Applicant: THE AUTOMATION PARTNERSHIP (CAMBRIDGE) LIMITED, Royston Hertfordshire (GB)

(72) Inventors: Mark VARLEY, Hardwick (GB); Sean SULLIVAN, Baldock (GB); Adrian Neil BARGH, Cambridge (GB)

(21) Appl. No.: 18/575,217

(22) PCT Filed: Jul. 15, 2022
(86) PCT No.: PCT/EP2022/069897

§ 371 Date: Dec. 28, 2023

(30) Foreign Application Priority Data
Jul. 16, 2021 (EP) 21186246.1

(51) Int. Cl. C12M 1/00; C12M 1/06; C12M 1/34; C12M 3/00

(52) U.S. Cl. CPC C12M 23/28; C12M 23/26; C12M 23/46; C12M 27/02; C12M 41/12

(57) ABSTRACT

A consumable (1) for a bioreactor system is provided. The consumable comprises a plurality of bioreaction vessels (2), each suitable for holding the contents of a bioreaction. The plurality of bioreaction vessels are provided as a single-piece construction, and each bioreaction vessel includes an agitator (4) configured to agitate the contents of the respective bioreaction vessel. Each agitator has a stirring end which extends into the bioreaction vessel and a drive end.

This application claims priority from EP 21186246.1 filed 16 Jul. 2021, the contents and elements of which are herein incorporated by reference for all purposes.

### FIELD OF THE INVENTION

The present invention relates to a bioreactor system consumable comprising a plurality of bioreaction vessels.

### BACKGROUND

Bioreactor systems are used to closely control the environments of cell culture solutions. In particular, the mixing of liquid and cells is critical to improving homogeneity within a bioreactor and hence control of the local environment to which cells within the bioreactor are exposed. To improve the ratio of success and speed to market of new biopharmaceutical products, there is a need to screen larger numbers of potential cell clones in small-scale production-like processes using bioreactor systems. This ensures the most suitable clones can be successfully identified and taken forward to a larger-scale development process.

Bioreactor systems with a plurality of small-scale bioreactors can be used to mimic larger scale cell culture environments. Ideally, when studying clone properties, cell culture conditions are uniform across the plurality of bioreactors to ensure that any differences observed in the produced cell clones can be attributed to the properties of the clones and not to individual bioreactors. For instance, stirring speed, temperature, DO (dissolved oxygen), pH set-point, allowable gas flows, feeding strategy and glucose set-point should be identical from bioreactor to bioreactor. The cell culture conditions should also be uniform within each individual bioreactor.

EP 2270129 A proposes a microscale bioreactor system comprising multiple single-use bioreaction vessels for containing cell cultures. An impeller-style agitator is provided for every bioreaction vessel to stir the contents of the vessel. At the beginning of an experiment each bioreaction vessel is individually loaded into the system by an operator. Furthermore, each agitator is manually orientated and connected to an external drive mechanism. This can be laborious and time consuming. More generally, the small size of the components and the large numbers of small parts mean that such microscale bioreactor systems are challenging to assemble and manufacture, leading to increased scrappage due to assembly errors.

Therefore, a bioreactor system is desired which is easier to assemble, efficient to load and unload between experiment runs, and provides uniform and controlled culture conditions.

### SUMMARY OF THE INVENTION

Accordingly, in general terms the present invention provides a consumable for a bioreactor system, the consumable comprising:
a plurality of bioreaction vessels, each suitable for holding the contents of a bioreaction,
wherein each bioreaction vessel includes an agitator configured to agitate the contents of the respective bioreaction vessel, each agitator having a stirring end which extends into the bioreaction vessel and a drive end.

In a first aspect, the present invention provides a consumable for a bioreactor system, the consumable comprising:
a plurality of bioreaction vessels, each suitable for holding the contents of a bioreaction,
wherein the plurality of bioreaction vessels are provided as a single-piece construction, and each bioreaction vessel includes an agitator configured to agitate the contents of the respective bioreaction vessel, each agitator having a stirring end which extends into the bioreaction vessel and a drive end.

Advantageously, such a system reduces the assembly time involved to set-up a bioreactor system and improves the efficiency of the turn-around between experiment runs.

Optional features of the invention will now be set out.

The consumable may further comprise a drive mechanism connected or connectable to the drive ends of the agitators of the plurality of bioreaction vessels and configured to move the agitators with identical synchronised motions. The identical synchronised motions are preferably rotary synchronised motions, but can be other motions, such as linear reciprocating synchronised motions. Such an arrangement reduces the number of connections to be made by an operator when setting up a bioreaction system and avoids the need to manually position each agitator. The consumable may be provided to a user as a single unit (i.e. with the drive mechanism already connected to the drive end) which may be loaded into a bioreaction system. Alternatively, the consumable may be provided to a user as a two-part kit wherein one part comprises the bioreaction vessels and agitators, and the other part comprises the drive mechanism which may only be connected to the agitators upon loading to the bioreaction system.

According to another option, a drive mechanism connectable to the drive ends of the agitators of the plurality of bioreaction vessels and configured to move the agitators with identical synchronised motions may be provided as a permanent (i.e. non-consumable) component of a bioreactor system into which the consumable is loaded. For example, such a drive mechanism can be incorporated into a clamp plate of the bioreactor system, the clamp plate being clampable to the top of the consumable and containing passages which thereby form respective sealed gas connections with, e.g. outgassing ports for exhausting gas from the vessels and/or gas inlet ports for introducing gas to the vessels. Such sealed gas connections are discussed further below. Under this option, the drive mechanism is removable from the drive ends of the agitators when the clamp plate is removed from the consumable.

The plurality of bioreaction vessels may be arranged in one or more rows and the drive mechanism (whether a part of the consumable or a non-consumable component of the bioreactor system) may be a stirrer bar extending adjacent to and parallel with the one or more rows. Such an arrangement helps to ensure that each agitator receives the same driving motion, and is simpler to manufacture and has fewer moving parts than individual, dedicated drive mechanisms driving each vessel. In addition, having the vessels arranged in rows is compatible with a modular system in which one or more further consumables may be mounted adjacent a first consumable, all with parallel rows (and stirrer bars if applicable), to increase in an orderly fashion the number of available vessels.

More generally, in a second aspect, the present invention provides a consumable for a bioreactor system, the consumable comprising:
a plurality of bioreaction vessels arranged in one or more rows, each vessel being suitable for holding the contents of a bioreaction,
wherein each bioreaction vessel includes an agitator configured to agitate the contents of the respective bioreaction vessel, each agitator having a stirring end which extends into the bioreaction vessel and a drive end; and
wherein the consumable further comprises a stirrer bar which extends adjacent to and parallel with the one or more rows, which is connected or connectable to the drive ends of the agitators of the plurality of bioreaction vessels, and which is configured to move the agitators with identical synchronised motions.

Further optional features of the invention will now be set out. These are applicable singly or in any combination with any aspect of the invention.

Typically the stirring end of each agitator extends downwards into the bioreaction vessel from its drive end.

The drive mechanism may include an interface to an external drive linkage which allows connection to an external source of motive power provided in the bioreactor system. This facilitates integration of the consumable with the system.

The stirring ends of the agitators may be paddles. Such an arrangement is compatible with an orbital stirring motion of the paddles, which can reduce or avoid relative sliding and/or rotary motion between parts in the bioreaction vessels. This in turn results in less wear at the parts. Reducing the wear of parts in the bioreaction vessels reduces potential contamination of the bioreactions by micro-particles released by the wearing parts. Moreover, reduced part wear ensures the agitators are less likely to break and the agitation effects produced by the agitators are uniform from the beginning of an experiment to the end of the experiment. Top-mounted paddles in particular can avoid having parts where there is relative sliding and/or rotary motion within the contents of the bioreaction, thereby avoiding cell damage from such motion. Furthermore, agitators which are paddles present a similar agitation surface to the bioreaction fluid at all depths, irrespective of how far the paddle is submerged in the fluid. Therefore, the paddle agitators can accommodate a larger range of fluid volumes in the bioreaction vessel without causing unwanted foam effects or differing surface agitator properties even when the bioreaction vessel is only partially full. In contrast, impeller agitators should be fully submerged to reduce foaming and cell damage.

Conveniently, the paddle agitators may be connected to the drive mechanism at their drive ends via respective ball and socket joints, or via respective living hinges. Both types of joint can allow orbital motion (a form of rotary motion) at the drive ends which provides corresponding orbital motion of the paddles. Living hinges allow the agitators and drive mechanism to be formed as one part (e.g. a single injection moulding), reducing the number of manufacturing steps.

The paddles may be tapered such that the widths of the paddles decrease with increasing distance from their drive ends. Such a tapered paddle design can prevent the distal ends of the paddles from touching the side walls of the bioreaction vessels where the radial displacement of the paddles is at a maximum during orbital motion. Therefore, the end of paddle can extend closer to the bottom of the bioreaction vessel, reducing the minimum volume of bioreaction fluid which is required in the vessel. Alternatively or additionally, the paddles may comprise holes, slots and/or additional paddle surfaces to improve fluid mixing in the bioreaction vessels.

Each agitator typically has a pivot point for its synchronised motion between its drive end and its paddle. Each bioreaction vessel may further include a flexible element around the respective agitator at the pivot point, the flexible element providing a seal which closes off the bioreaction vessel at the agitator. Thus, the flexible element can help to prevent off-gas or fluid from the bioreaction escaping the bioreaction vessel at the entry point of the agitator to the bioreaction vessel, promoting a controlled environment inside the vessel. Conveniently, it does this while being located at a position along the agitator where the amplitude of the agitator's motion is at a minimum, helping to reduce the amount of relative motion between parts. This in turn results in less wear at the parts, thereby reducing contamination of the bioreaction. The flexible element may be made of a thermoplastic elastomer or silicone, or any other suitable elastomeric material that is biocompatible, able to form a seal, and can be sterilised by irradiation, autoclaving, EtO (Ethylene Oxide) or using a similar method.

The flexible element may be fastened to the bioreaction vessel, for example using a separate retaining element which clamps the flexible element in place. The flexible element may be keyed to the bioreaction vessel to prevent rotation of the flexible element relative to the vessel. Conveniently, the flexible element may be over-moulded around the agitator.

As an alternative to paddles, the stirring ends of the agitators may be impellers. In this case the identical synchronised motions of the agitators are rotary synchronised motions which turn the impellers about respective axes thereof. The agitators may have one or more blades on their stirring ends and may be configured to rotate in the vessel such that the contents of the bioreaction vessel are stirred by the blades. Particularly when the stirring ends of the agitators are impellers, it may be convenient for the consumable to be provided to a user as a two-part kit wherein one part comprises the bioreaction vessels and agitators, and the other part comprises the drive mechanism which is then connectable to the drive end of each agitator. When the drive mechanism is a stirrer bar, this may provide a circulating motion which is converted by respective mechanisms, for example pin wheel mechanisms, into rotation of the impellers about their axes. Such mechanisms can be incorporated in a clamp plate of the bioreactor system as further permanent (i.e. non-consumable) components of the system.

The bioreaction vessels may comprise respective outgassing ports for exhausting gas from the bioreaction. Each outgassing port may include a tortuous gas path for controlled gas exhaust therethrough. For example, tortuous gas path can help a slight positive pressure to be maintained in the vessels relative to the outside, whereby the expulsion rate and mixing of exhaust gases can be controlled, and ingress of external air by back flow along the path is discouraged. Moreover, the tortuous gas path can ensure that any incoming gases mix with existing gases in a head space formed between the top surface of the bioreaction and a lid of the vessel instead of directly exiting through the outgassing port before this mixing can occur. The bioreaction vessels may further comprise respective gas inlet ports for introducing gas into the vessels.

As an alternative to a tortuous gas path being formed in the consumable, the outgassing ports of the bioreaction vessels may form respective sealed gas connections to a permanent (i.e. non-consumable) component of a bioreactor system into which the consumable is loaded, and the tortuous gas paths can be formed in that component. For example, a clamp plate of the bioreactor system may contain exhaust passages which, when the clamp plate is clamped to the top of the consumable, form respective sealed gas connections with the outgassing ports for exhausting gas from the vessels. The exhaust passages can then include tortuous gas paths for controlled gas exhaust therethrough. An advantage of this arrangement is that the tortuous gas paths can be located in an environment (e.g. above the vessels and in a zone that is at a higher temperature than that of the gas head space which forms in the upper regions of the vessels) which is less susceptible to formation of condensate in the paths, such condensate formation providing a mechanism by which liquid can be lost from the vessels. When the bioreaction vessels further comprise respective gas inlet ports for introducing gas into the vessels, the clamp plate may further contain inlet passages which, when the clamp plate is clamped to the top of the consumable, form respective sealed gas connections with the gas inlet ports for the gas introduction.

As noted above, the bioreaction vessels may comprise respective outgassing ports for exhausting gas from the bioreaction and respective gas inlet ports for introducing gas into the vessels. With such an arrangement, within each vessel, the respective outgassing port and respective inlet port are preferably configured such that gas introduced through the inlet port mixes substantially completely with gas already present in the head space which forms in the upper region of the vessel before exiting via the outgassing port. Thus this port configuration avoids gas flow in a short-circuit directly from the inlet port to the outgassing port. For example, when the stirring ends of the agitators are impellers and the agitators are thus rotated around impeller axes, this rotation locally disturbs the airflow in the head space, and the ports can be configured so that the gas introduced through the inlet port is delivered into this disturbed airflow to promote its mixing.

When the bioreaction vessels comprise respective outgassing ports and/or respective gas inlet ports, the vessels may be arranged in one or more rows, and may be further arranged in side-by-side mirrored pairs along the or each of the rows. Conveniently, when the bioreactor system has a cooling sub-system (discussed below in respect of the third aspect) configured to promote condensation of off-gases in the gas head space which forms in upper regions of the vessels, each side-by-side mirrored pair can then share a respective cooling element of the cooling sub-system, the cooling element being located in the upper region of the channel formed in space between the two vessels of the pair. In this way, one cooling element can produce the same thermal effect on both vessels of the pair, and thus cooling of the off-gases can be performed with fewer cooling elements. Also fewer cooling elements are required, which reduce the cooling power demand.

Additionally or alternatively when the bioreaction vessels comprise respective outgassing ports and/or respective gas inlet ports, the vessels may be arranged in parallel rows on opposite sides of a centre line, and may be further arranged in mirrored pairs with respect to their ports across the centre line. When the consumable or the wider bioreactor system has a stirrer bar, this can conveniently extend along the centre line.

The plurality of the bioreaction vessels may be rigid containers. Conveniently they can be formed of injection moulded or blow moulded plastic. Typically they are clear-walled to allow their contents to be viewed.

In a third aspect, the present invention provides a bioreactor system comprising the consumable of the first or second aspect.

The bioreactor system may further comprise a non-consumable clamp plate clampable to the top of the consumable and containing passages which thereby form respective sealed gas connections with, e.g. outgassing ports for exhausting gas from the vessels and/or gas inlet ports for introducing gas to the vessels. Such passages which form respective sealed gas connections with the outgassing ports can include tortuous gas paths for controlled gas exhaust therethrough. Passages which form respective sealed gas connections with the gas inlet ports typically join in the bioreactor system to one or more sources of introduced gas, e.g. air or oxygen, for controlling the gas composition in the head spaces of the vessels, while passages which form respective sealed gas connections with the outgassing ports typically vent to atmosphere.

The bioreactor system may further comprise a source of motive power for moving the agitators of the consumable. The bioreactor system may include a drive linkage which interfaces at one end to a drive mechanism (e.g. a stirrer bar) connected or connectable to the drive ends of the agitators of the plurality of bioreaction vessels and configured to move the agitators with identical synchronised motions. Such a drive mechanism can be a drive mechanism of the consumable or a drive mechanism which is a permanent, non-consumable component of the bioreactor system into which the consumable is loaded. For example, the drive mechanism can be incorporated as a non-consumable component into a clamp plate of the bioreactor system. The drive linkage connects at another end to the source of motive power to transmit the motive power to the drive mechanism. In the case of a stirrer bar drive mechanism incorporated into a clamp plate as a non-consumable component, the clamp plate can further incorporate respective non-consumable mechanisms, e.g. pin wheel mechanisms, for converting circulating motion of the stirrer bar into rotation of impeller agitators about their axes.

The bioreactor system may further comprise a control unit for programmably controlling the movement of the agitators.

The bioreactor system may further comprise a cooling sub-system configured to promote condensation of off-gases in the gas head space which forms in upper regions of the vessels. For example, the system may have cooled projections e.g. cooled by Peltier cooling elements, which extend in the upper regions of the channels formed in spaces between the vessels. Conveniently, the cooling sub-system can be arranged to reject heat into a clamp plate of the bioreactor system. When such a clamp plate contains passages which form respective sealed gas connections with outgassing ports of the vessels, the rejected heat can raise the temperature of these passages, thereby discouraging the formation of condensation within them. This can be particularly beneficial when the passages include tortuous gas paths for controlled gas exhaust therethrough,

Additionally or alternatively, the bioreactor system may further comprise a heating sub-system configured to perform temperature control of bioreaction liquid contained within lower regions the vessels. For example, the system may have a solid state heating block providing heated projections which extend into lower regions of the channels formed in the spaces between the vessels.

Preferably, the bioreactor system comprises both the cooling sub-system and the heating sub-system, as this enhances the ability to closely control the conditions of the bioreaction.

The bioreactor system may comprise plural of the consumables of the first or second aspect. A clamp plate of the system can then clamp more than one consumable, and correspondingly can incorporate more than one drive mechanism. The bioreactor system may have a respective cooling sub-system and/or a respective heating sub-system for each consumable.

More generally, in a fourth aspect, the present invention provides a bioreactor system having one or more loading stations suitable for loading with one or more of the consumables of the first or second aspect. When loaded with the one or more of the consumables, the bioreactor system of the fourth aspect can thus become the bioreactor system of the third aspect Accordingly, the bioreactor system of the fourth aspect may have one or more of the aforementioned clamp plates clampable to the top of the consumable(s) when loaded in the station(s). Likewise, the bioreactor system of the fourth aspect may have at the or each loading station a respective aforementioned cooling sub-system and/or a respective aforementioned heating sub-system.

The present invention includes combination of any of the aspects and optional features described, except where such a combination is clearly impermissible or expressly avoided.

### SUMMARY OF THE FIGURES

Embodiments of the invention will now be described by way of example with reference to the accompanying drawings in which:

FIG. 1 shows a perspective view of a first embodiment of a consumable for a bioreactor system;

FIGS. 2A to 2D show respectively a plan view, a side elevation view, a sectional view along plane A-A, and a sectional view along plane B-B of the consumable of FIG. 1;

FIG. 3A shows a sectioned perspective view of the consumable of FIG. 1, and FIG. 3B shows a detailed view of section region C of FIG. 3A;

FIG. 4A shows a variant of a stirrer bar of the consumable of FIG. 1, and FIG. 4B shows a detailed view of region D of FIG. 4A;

FIGS. 5A and 5B show a perspective view and a plan view respectively of a paddle agitator providing a linear reciprocating motion;

FIGS. 6A and 6B show a perspective view and a plan view respectively of a paddle agitator providing an orbital motion;

FIG. 7 shows variants of a paddle agitator for use in the consumable of FIG. 1;

FIG. 8A shows a further plan view of the consumable of FIG. 1, and FIG. 8B shows a detailed view of region E of FIG. 8A;

FIG. 9 shows a further side view of the consumable of FIG. 1;

FIG. 10 shows a perspective view of a second embodiment of a consumable for a bioreactor system, a drive mechanism of the consumable not being shown;

FIGS. 11A to 11D show respectively a plan view, a side elevation view, a sectional view along plane B-B, and a sectional view along plane C-C of the consumable of FIG. 10, again a drive mechanism of the consumable not being shown;

FIG. 12 shows a perspective view of part of the consumable of FIG. 10 with impeller agitators removed from some bioreaction vessels, and with vessel lids, a handle and a drive mechanism of the consumable not being shown;

FIGS. 13A to 13C show respectively a plan view, an elevation view and a sectional view along plane F-F of a variant of two of the consumables of FIG. 10 joined together in a modular fashion;

FIG. 14A shows a perspective transparent view of an impeller connector and FIG. 14B shows a perspective view of an interfacing connector which connects a drive shaft to the impeller connector

FIG. 15 shows a perspective view of a bioreactor system loaded with an example of a third embodiment of a multi-parallel, single-use consumable;

FIGS. 16A and 16B show respectively a side elevation view, and a sectional view along plane B-B of the bioreactor system of FIG. 15, clamp plates of the system not being shown;

FIG. 17 is a cross-section through two neighbouring vessels of the consumable of the third embodiment loaded in the bioreactor system of FIG. 15; and

FIG. 18 is a transparent perspective view of part of the vessel and lid of the consumable of the third embodiment.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference

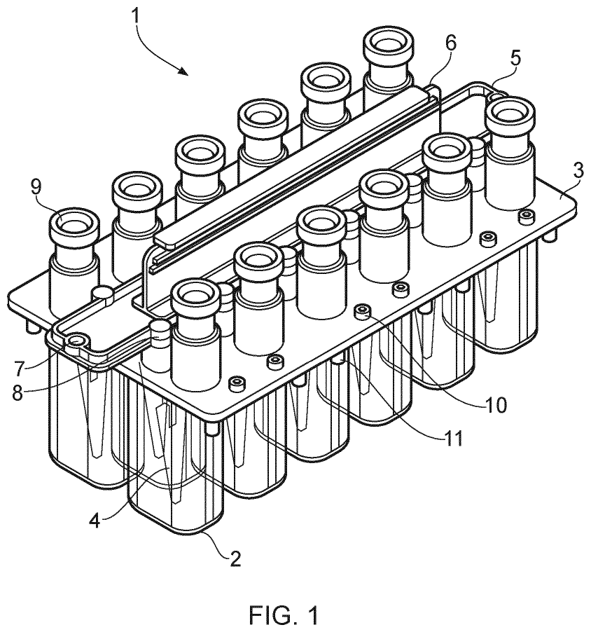

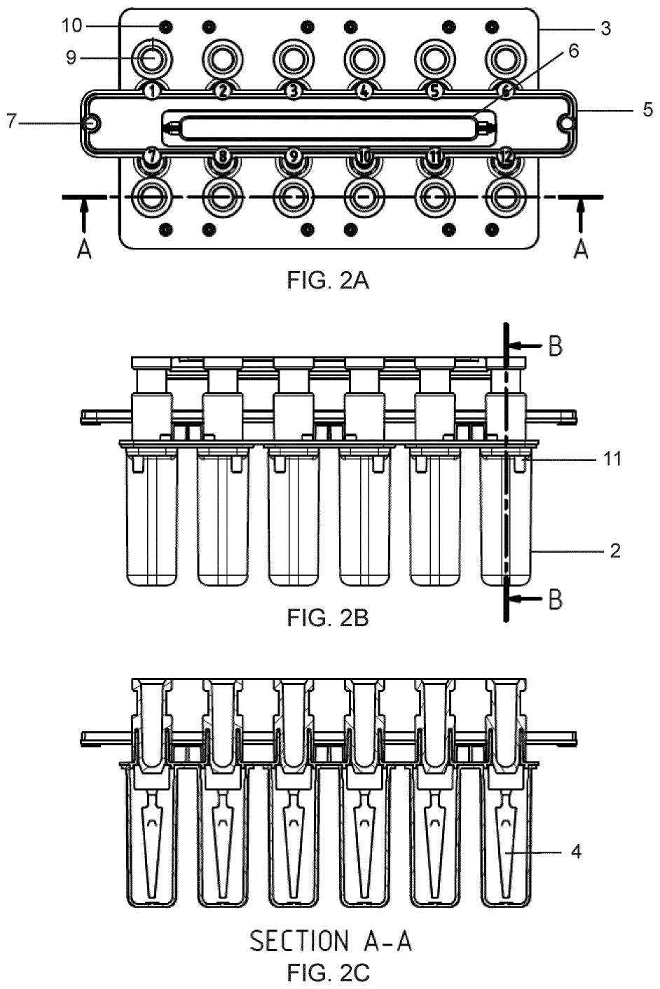

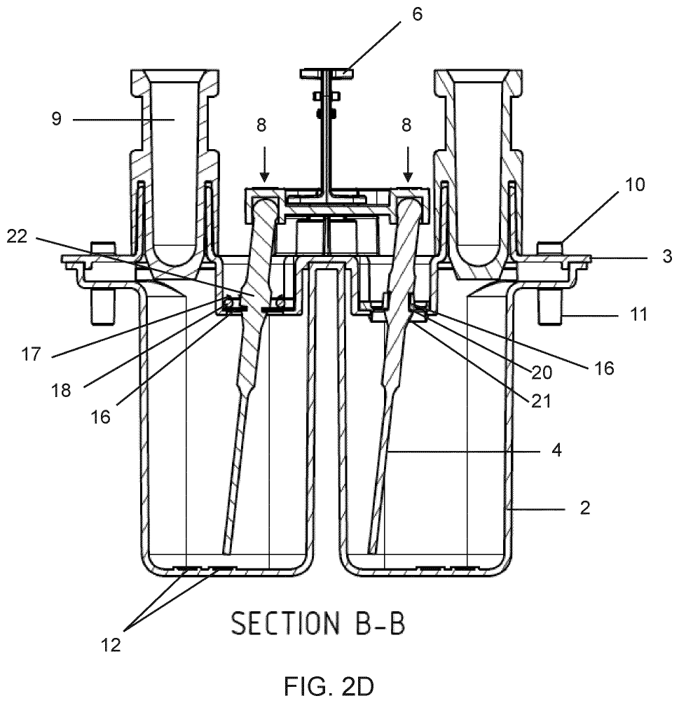

FIG. 1 shows a perspective view of a first embodiment of a multi-parallel, single-use consumable for a bioreactor system, and FIGS. 2A to 2D show respectively a plan view, a side elevation view, a sectional view along plane A-A, and a sectional view along plane B-B of the consumable of FIG. 1. The consumable 1 comprises a plurality of rigid bioreaction vessels 2 (two parallel rows are shown, each row having six vessels) suitable for holding the contents of a bioreaction. Each bioreaction vessel includes an agitator 4 for agitating the contents of the respective vessel, the agitator having a stirring end in the form of a paddle which extends into the bioreaction vessel and a drive end outside the vessel. The bioreaction vessels are a single piece construction, and conveniently may be formed of injection moulded or blow moulded plastic. The vessels are configured to be serviceable by a robotic liquid handler. Alternatively, they can be serviced manually.

The bioreaction vessels 2 are closed by a lid 3, with the agitators 4 extending through respective apertures formed in the lid. Typically, the lid is ultrasonically welded to the vessels. However, alternative arrangements may be possible. For example, each vessel or subset of vessels may have its own lid. In the embodiment shown, each vessel is configured to support a bioreaction working volume of between 0.5 mL and 3.5 mL and the consumable is anticipated to be in continuous use for bioreactions lasting typically 2 weeks but could extend up to 4 weeks. However, the vessels may be sized to support other working volumes, and the consumable adapted to last for shorter or longer durations.

The consumable 1 allows an operator to load the bioreaction vessels 2 in batches. Variants of the consumable may have any number of the vessels and/or different arrangements of the vessels within the consumable. For example, a consumable with 24 or 36 bioreaction vessels may be provided for larger scale experiments. Similarly, the 12 bioreaction vessels may be provided in the 2 by 6 arrangement shown in FIG. 1, or in a 3 by 4 arrangement. The multiple vessels form a “rack” which can be loaded in one action, and which has a wide footprint that ensures the consumable is stable when standing on a bench.

A drive mechanism in the form of a stirrer bar 5 is connected to the drive ends of the agitators 4 by respective ball and socket joints 8. The stirrer bar extends between and parallel to the rows of vessels 2 so that each agitator receives the same driving motion. The stirrer bar has holes 7 at both ends which interface to an external drive linkage (not shown) for connecting to an external source of motive power of the bioreactor system, such as an electric motor. The linkage moves the stirrer bar back and forth or with a circulating motion which induces in the ball and socket joints respectively a linear reciprocating motion or an orbital motion. As discussed in more detail below, this motion of the joints then induces a corresponding motion of the paddles, thereby stirring the contents of the bioreaction. The paddles reduce the shear stress on the cells of the bioreaction and provide good stirring conditions across a wide range of working volumes.

The stirrer bar is 5 provided as part of the consumable 1 and links all of (or a subset of) the agitators 4 in the consumable together. Therefore, the induced motions of the paddles are identical synchronous motions. This ensures consistency in the bioreaction environments across the plurality of bioreaction vessels. Furthermore, such an arrangement is simpler to manufacture and has fewer moving parts than individual, dedicated drive mechanisms driving each agitator. A programmable control unit (not shown) controls the electric motor and linkage, and thereby controls the type, speed and duration of the paddle motion.

A handle 6 extends upwards from the lid 3 through a slot in the stirrer bar 5. Moreover, a flange protrudes from both sides of the handle above the slot in the stirrer bar to prevent the stirrer bar detaching from the consumable. Typically, a unique identifier such as a bar code is included on the handle to identify the batch of bioreactions contained in the bioreaction vessels 2. Each bioreaction vessel 2 includes a capped sample port 9 to allow fluid to be added to the vessel or samples to be taken from the vessel, e.g. by a pipette. Each sample port can have its own separate cap, as shown in FIG. 1. Alternatively, groups of sample ports can share integrated caps, e.g. in the example of FIGS. 1 and 2 each row of six vessels 2 can have a six-way integrated cap that allows all the vessels of the row to be opened and closed by respectively removal and fitting of the single integrated cap. Sensors 12 are integrated in the base of each vessel to allow properties of the bioreactions to be monitored (for example, pH or DO).

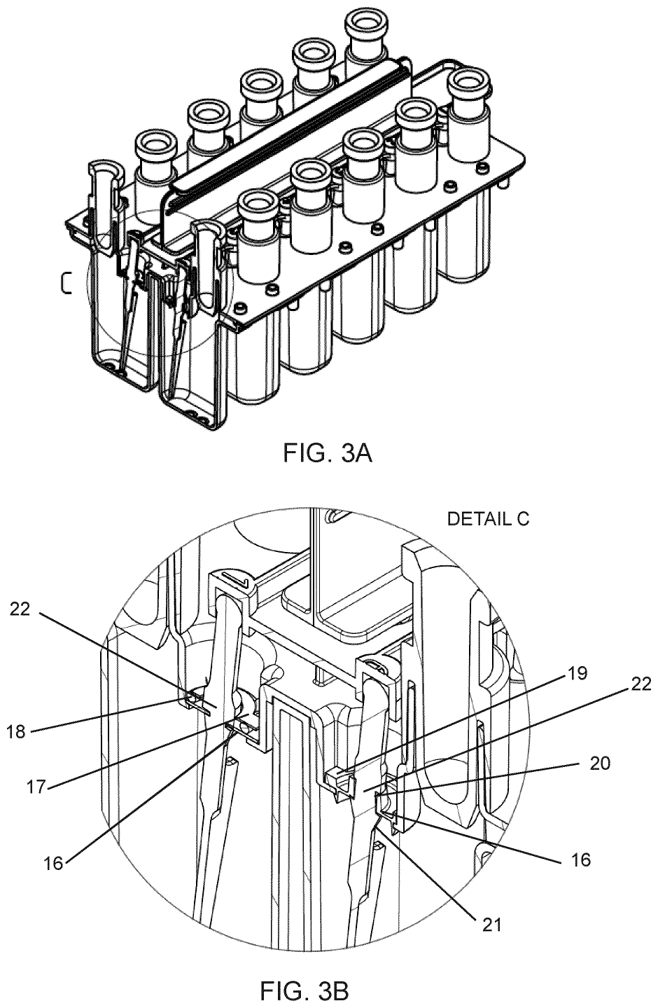

FIG. 3A shows a sectioned perspective view of the consumable of FIG. 1, and FIG. 3B shows a detailed view of section region C of FIG. 3A. Flexible elements 16 are provided where the agitators 4 extend through the respective apertures formed in the lid 3. Each flexible element provides a deformable, sterile seal between its agitator and the lid 3 that helps to prevent off-gases from the bioreaction escaping through the aperture in the lid. Thus, a controlled gas composition can be maintained in the head space between the bioreaction fluid and the lid. The seal also helps to prevent contamination of the bioreaction from external agents. Moreover, the flexible element also helps to maintain the vertical position of the agitator in its vessel, and also constrains any sideways motion at the level of the flexible element. In this way, when the agitator is moved by the stirrer bar 5, the flexible element deforms slightly to accommodate the movement while maintaining the seal. The flexible element is able to do this because it effectively defines a pivot point 22 of the agitator between on one side the driven motion of the ball and socket joint and on the other side the corresponding induced motion of the paddle.

The flexible element 16 may be made of silicone or polyethylene or any other suitable elastomer material which is biocompatible and sterilisable. An index tab 19 is provided on the flexible element for keying to a respective slot formed the lid. This prevents the flexible element, and the agitator 4, from rotating about the centre of the respective aperture. Conveniently, the whole stirring mechanism can be sterilised e.g. using irradiation, autoclaving or EtO.

The sections of FIGS. 2D, 3A and most clearly 3B show two different arrangements to secure the agitators 4 and the flexible elements 16 to the lid 3. A first arrangement is shown in the left vessel wherein the flexible element 16 is a planar elastomeric annulus resting at its outer perimeter on a rim formed by the lid 3 around the aperture, while the inner perimeter of the annulus fits snugly in a circumferential groove formed in the agitator. An O-ring 18 is provided above the flexible element to perfect the seal, and is secured in place by a retaining element 17 (which also has an index tab to prevent its rotation about the agitator).

A second locking arrangement is shown in the right vessel 2 wherein the agitator 4 is held in place by just the flexible element 16. In this case, the circumferential groove 20 formed in the agitator is more substantial, and the inner perimeter of the flexible element is sized to match. Moreover, the outer circumference of the flexible element has a groove formed therein which receives the edge of the rim formed by the lid. This arrangement is more suitable for forming the flexible element 16 as an over-mould around the agitator 4.

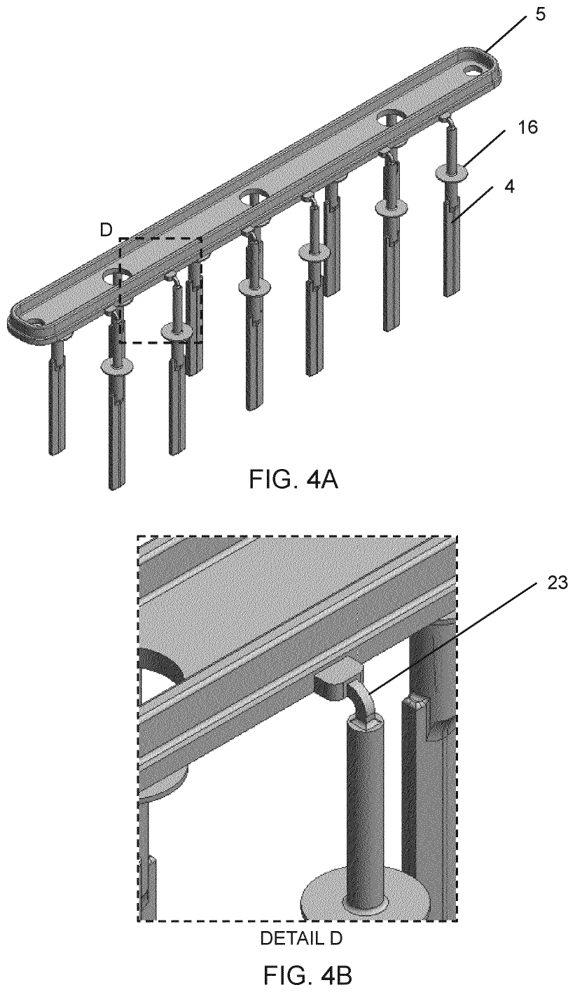

FIG. 4A shows a variant of the stirrer bar 5 of the consumable of FIG. 1, in which the agitators are connected to the stirrer bar using living hinges 23 instead of ball and socket joints. FIG. 4B shows a detailed view of region D of FIG. 4A. In this variant, the agitators 4 and stirrer bar 5 may be formed as one part, for example by injection moulding, reducing the number of manufacturing steps.

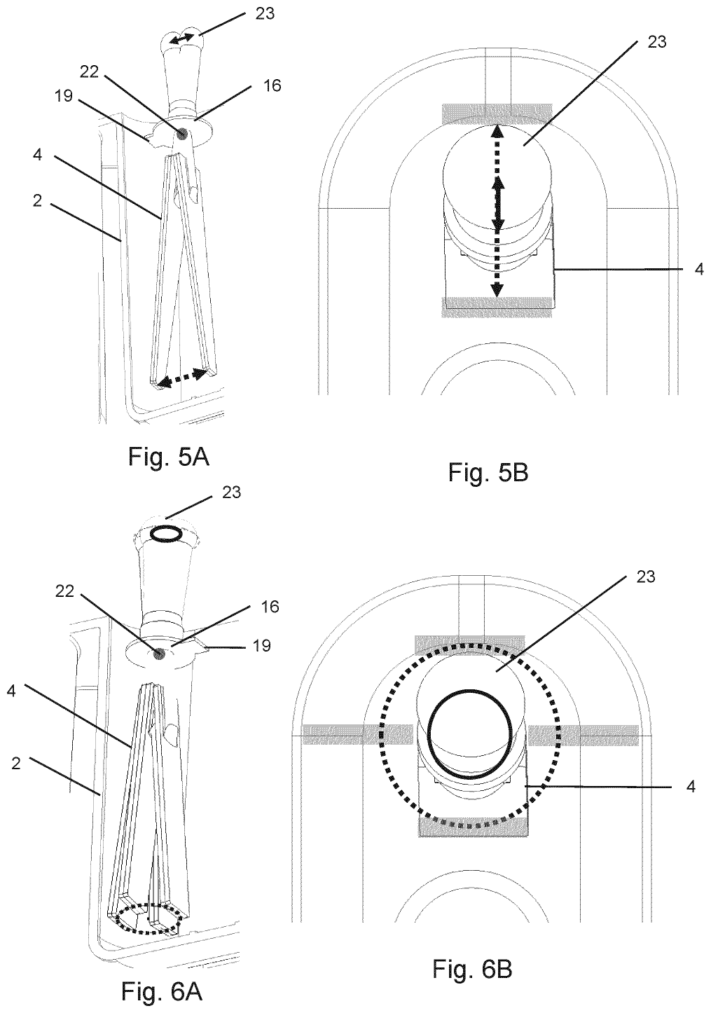

FIGS. 5A and 5B show a perspective view and a plan view respectively of a paddle agitator 4 undergoing a linear reciprocating motion. The drive end of the agitator, corresponding to the ball and socket joint 23, is driven via a back and forth movement of the stirrer bar 5 along a linear reciprocating path, as indicated by the solid arrowed lines. The agitator pivots about the pivot point 22 and the distal end of the paddle on the opposite side of the pivot point follows a corresponding linear reciprocating path, indicated by the dashed arrowed lines at the bottom of the bioreaction vessel 2. The ratio of the amplitudes of the reciprocating paths at the ball and socket joint 23 and the distal end of the paddle depends on the ratio of the distances of the ball and socket joint and the distal end of the paddle to the pivot point.

FIGS. 6A to 6B show a perspective view and a plan view respectively of a paddle agitator 4 undergoing an orbital motion. The drive end of the agitator, corresponding to the ball and socket joint 23 is driven via a circulating movement of the stirrer bar 5 along a circular orbit indicated by the solid arrowed line. The distal end of the paddle describes a corresponding circular orbit at the bottom of the bioreaction vessel 2, as indicated by the dotted arrowed line. The ratio of the diameters of the circular orbits at the ball and socket joint 23 and the distal end of the paddle depends on the ratio of the distances of the ball and socket joint and the distal end of the paddle to the pivot point.

The motion of the paddles 4 is not limited to circular orbital or linear reciprocating motions. For example, the paddles may be driven in figure-of-eight or elliptical motions.

A benefit of paddle agitators 4 is that the motion of the paddles results in agitation of both the bioreaction fluid and the head space gas above the bioreaction fluid. Moreover, the paddles can be formed as single pieces, which do not require assembly of multiple parts. Typically, they are made of rigid plastic. Unlike conventional stirred impellers, the paddles avoid having relative sliding and/or rotary motion between parts that are immersed in the bioreaction fluid. They also enable a securing arrangement that is less susceptible to wear, rubbing, grinding and fatigue damage than conventional stirred impellers, helping to avoid the creation of wear particulates within the vessels 2 and reducing vibration and noise.

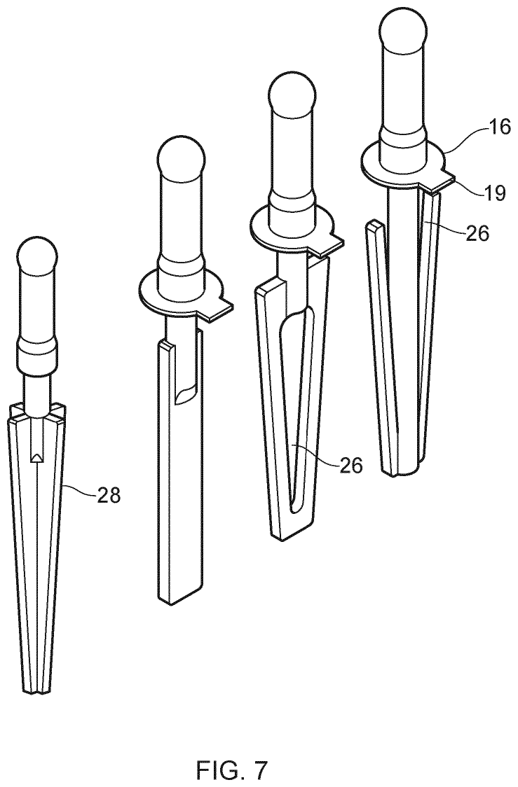

FIG. 7 shows variant shapes of the paddle agitator 4. The paddles may have one or more of: holes or slots 26 formed in the paddle, additional paddle surfaces 28, and tapered or non-tapered profiles. Different paddle geometries may be used to achieve a desired kLa (gas transfer) and shear profile for cells. As described above a tapered profile can accommodate a large range of fluid volumes in the vessel without causing unwanted foam affects or differing surface agitator properties. For example, cells in 0.5 mL of bioreaction fluid will see substantially the same forces as cells in 3.5 mL of fluid, as the paddle traverses the entire working volume. In contrast, with a conventional impeller, cells in different volumes of fluid can experience different conditions, and particularly if the fluid drops below the top level of the impeller to encourage foaming.

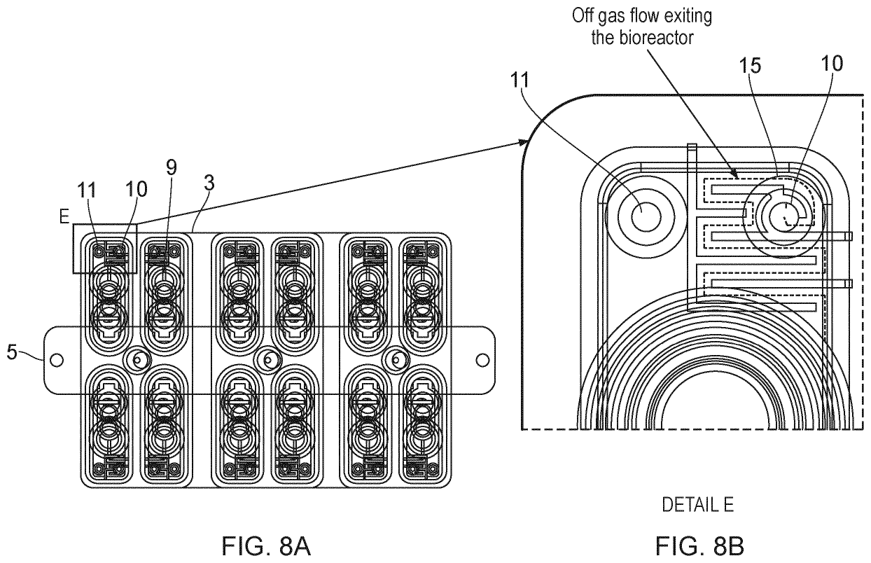

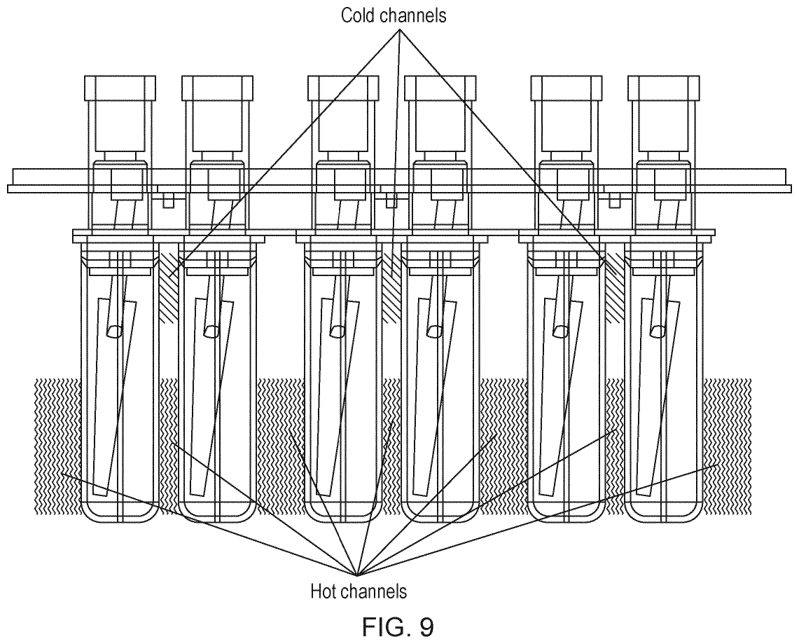

FIG. 8A shows a further plan view of the consumable of FIG. 1, and FIG. 8B shows a detailed view of region E of FIG. 8A including an exhaust channel 15. Each bioreaction vessel 2 includes such an exhaust channel to allow the exhaustion of gases (off-gases) which may be products of the bioreaction. Additionally, an inlet gas port 11 is provided for each vessel 2 for introducing gases to the vessel. Off-gas is expelled from the vessel into the exhaust channel 15 leading to an outlet gas port 10 in the lid 3. The exhaust channel follows a tortuous route to the outlet gas port. For instance, the exhaust channel may be long and narrow and have multiple bends. Such a tortuous route encourages a slight positive pressure in the vessel 2 to reduce diffusion of ambient air back into the vessel, reduces the diffusion rate of head space gasses from the vessel, and hence reduces evaporation of the bioreaction fluid. Furthermore, the exhaust channel allows the off-gases to be sensed as they exit the vessel. The exhaust channel may be inclined to allow the collection of any condensate back into the vessel 2. Alternatively or additionally, in some arrangements, the off-gases may be allowed to exhaust through gas escape routes provided by imperfect seals between the agitators 4 and the lid 3.

FIG. 9 shows a further side view of the consumable 1 of FIG. 1, illustrating cold and hot channels. Cooling elements can be provided in the bioreaction system at the cold channels to promote condensation of off-gases in the gas head space which forms in upper regions of the vessels, and preferably before entry into the exhaust channels 15 so that evaporation fluid losses are reduced. The bioreaction vessels 2 are arranged in side-by-side mirrored pairs along each of the two rows of vessels, and are also arranged in mirrored pairs across the centre line of the consumable. The side-by-side mirrored pairing ensures that the exhaust channels of each vessel in a pair can be arranged close to each other. Each side-by-side mirrored pair can also share a cooling element which produces the same thermal effect on both vessels of the pair, and by requiring fewer cooling elements imposes a lower power demand on the system. Thus cooling of the off-gases can be performed with fewer cooling elements. The hot channels meanwhile enable temperature-control of the contents of the bioreaction vessels by conduction of heat into the vessels from these channels, e.g. by loading the consumable into a solid state heating block having heated projections extending into the channels, or by providing heated airflows along the channels. Arranging the vessels in mirrored pairs across the centre line, as well as in side-by-side mirrored pairs, promotes reproducibility of thermal conditions throughout the vessels and accurate temperature control.

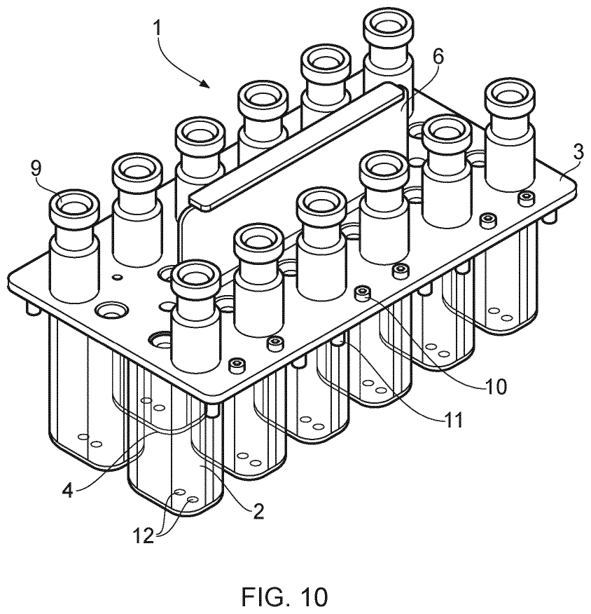

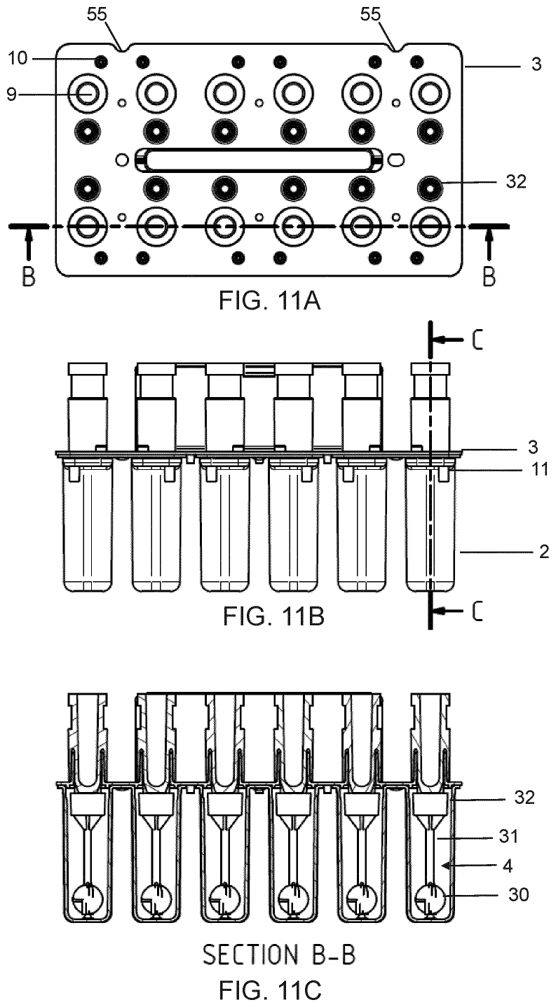

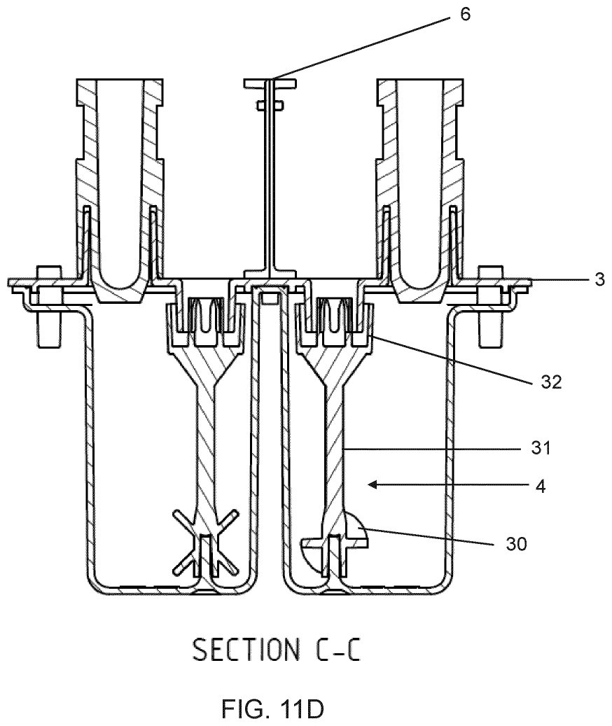

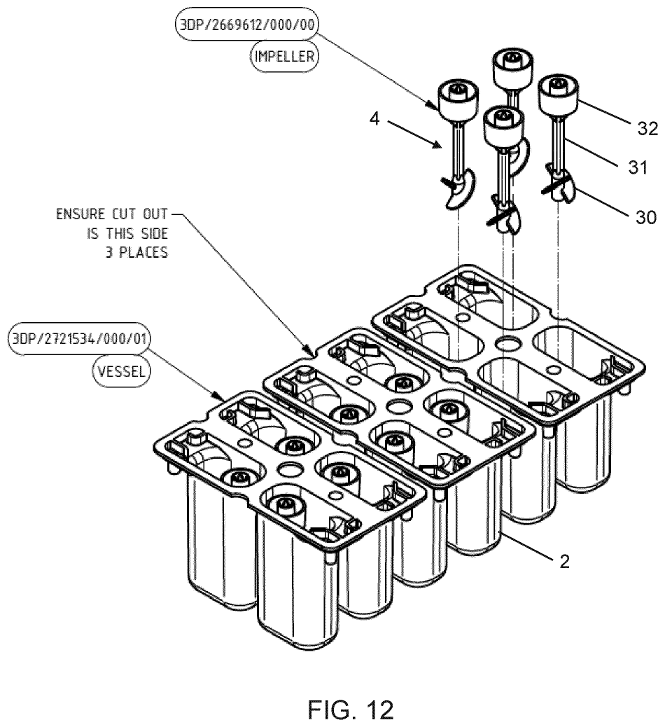

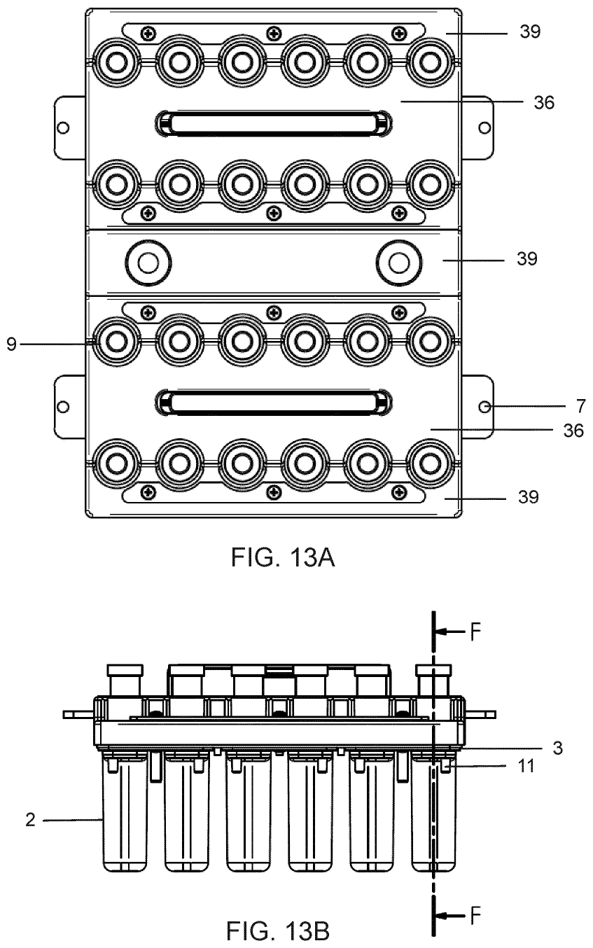

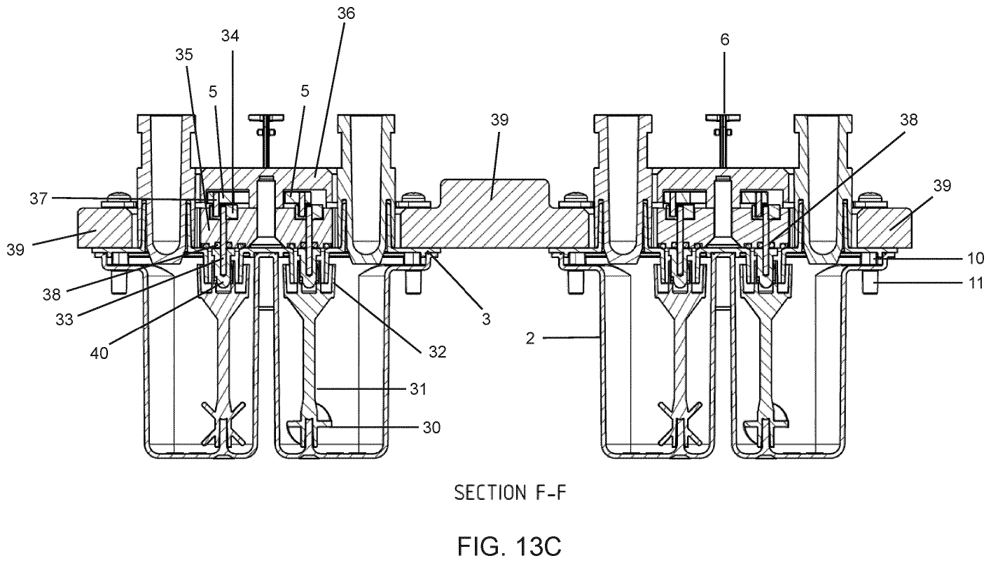

FIG. 10 shows a perspective view of a second embodiment of a consumable 1 for a bioreactor system, a drive mechanism of the consumable being omitted; FIGS. 11A to 11D show respectively a plan view, a side elevation view, a sectional view along plane B-B, and a sectional view along plane C-C of the consumable of FIG. 10; and FIG. 12 shows a perspective view of part of the consumable of FIG. 10 with agitators 4 removed from some bioreaction vessels 2. Additionally, FIGS. 13A to 13C show respectively a plan view, an elevation view and a sectional view along plane F-F of two of the consumables of FIGS. 10 to 12 joined together in a modular fashion. The consumable comprises a plurality of bioreaction vessels 2 closed by respective lids 3, as in the consumable of the first embodiment. Semi circular cut-outs 55 along an edge of the lid 3 mechanically interlock with the system hardware to ensure the consumable is always loaded in the correct orientation. Side plates 39 hold the lid 3 in place at the sides of the consumable and allow adjacent consumables to be joined together, as shown in FIGS. 13A to 13C. However, the agitators 4 of the second embodiment are impellers, each impeller comprising a shaft 31 extending into its respective bioreaction vessel, and blades 30 at the bottom end of the shaft for stirring the contents of the bioreaction. The top end of each shaft terminates in an impeller connector 32 which fits loosely around sides of a respective well formed in the lid. In this way the impeller connector 32 forms a labyrinth with the sides of the well which can catch any wear particulates formed by the drive mechanism, helping to prevent contamination of the content of the bioreaction vessel. The well has an aperture at its bottom through which a drive shaft 33 (described below) joins to the impeller connector 32. A further contrast with the consumable of the first embodiment is that rather than having all the bioreaction vessels 2 formed by one single piece construction, they are formed by several (three shown in FIG. 12) single piece constructions, each construction providing plural (four shown in FIG. 12) of the vessels. The single piece constructions are then located relative to each other by the lid 3.

In the embodiment shown, each vessel is configured to support a bioreaction working volume of between 1.5 mL and 3.5 mL.

A drive mechanism (shown best in FIG. 13C), in the form of a stirrer bar 5, is provided to rotate the impeller agitators 4 about the axes of their shafts 31. The stirrer bar 5 is held between stationary lower 35 and upper 36 clamp plates and is connected to each impeller by a respective pin wheel mechanism. More particularly, the stirrer bar has holes 7 at both ends which interface to an external drive linkage (not shown) for connecting to an external source of motive power of the bioreactor system. The linkage moves the stirrer bar with a circulating motion which induces the pin wheel mechanisms to rotate their respective impellers. A handle is connected directly to the lid 3 through aligned slots in the clamp plates and stirrer bar, the slot in the stirrer bar being sized so that handle does not interfere with the motion of the stirrer bar. Conveniently, the upper clamp plates 36 and the side plates 39 can be integrated as a single component. The stirrer bar 5, drive shafts 33, rotatable discs 34, lower clamp plates 35, upper clamp plates 36, pins 37, O-rings 38, side plates 39 and interfacing connectors 40 form a multi-use consumable part of the system which is suitable for repeat (although not indefinite) use, while other parts of the bioreactor system, such as the bioreaction vessels 2, lids 3, agitators 4, form a single-use consumable part of the system which is not intended for repeat use. In FIGS. 10 to 13, the multi-use consumable part is shown interfacing to two single-use consumable parts, but a multi-use consumable part such as this may be extended as needed to interface to any number of single-use consumable parts.

Each pin wheel mechanism comprises a pin 37 extending from the stirrer bar 5 into rotatable discs 34 situated in matching recesses formed in the lower clamp plate 35. When the stirrer bar is moved in a circulating motion, the rotatable discs are rotated by the pins. A drive shaft 33 connects each rotatable disc to its impeller via the respective impeller connector 32. Thus, the circulating motion of the stirrer bar, relative to the upper and lower stationary clamp plates, drives the rotatable discs, which in-turn rotates the drive shafts to turn the impellers. The drive shaft is connected to its respective impeller connector 32 by an interfacing connector 40.

O-rings 38 are provided around each drive shaft 33 in the lower clamp plate 35 to form an airtight seal around the drive shaft. This prevents off-gases from the bioreaction and gases which are maintained in the head space between the bioreaction fluid and the lid 3 from escaping through the lid 3.

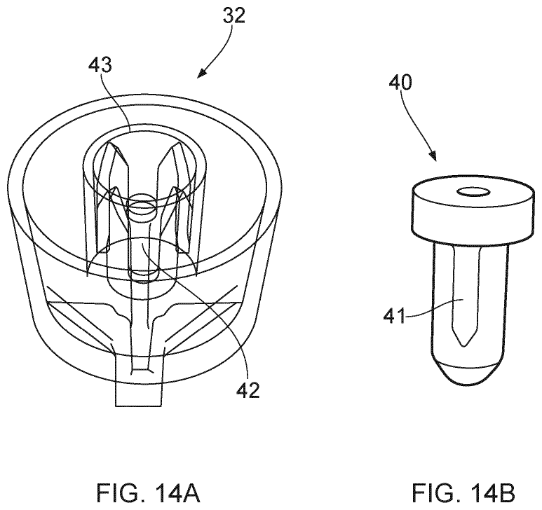

FIG. 14A shows a perspective transparent view of an impeller connector 32, and FIG. 14B shows a perspective view of an interfacing connector 40 at the end of the drive shaft 33 which connects the drive shaft to the impeller connector. The interfacing connector 40 inserts into a central receiving well 43 of the impeller connector 32. Side ridges 41 are provided on the interfacing connector which slide into corresponding slots 42 in the receiving well 43. Thus, when the interfacing connector 40 is rotated by the drive shaft 33, the impeller connector 32, and hence also the impeller 4, are rotated. The lower end of the interfacing connector 40 is tapered and leading edges of the side ridges are pointed to promote self-alignment of each interfacing connector 40 and its corresponding drive shaft 33 by its impeller connector 32 when the consumable is assembled. This self-aligning ability allows the consumable to be supplied to a user in two parts for assembly together by the user: one part comprising the vessels 2, impellers 4 and lid 3, and another part comprising the side plates 39 and drive mechanism (i.e. lower 35 and upper 36 clamp plates, stirrer bar 5, pin wheel mechanisms and drive shafts 33).

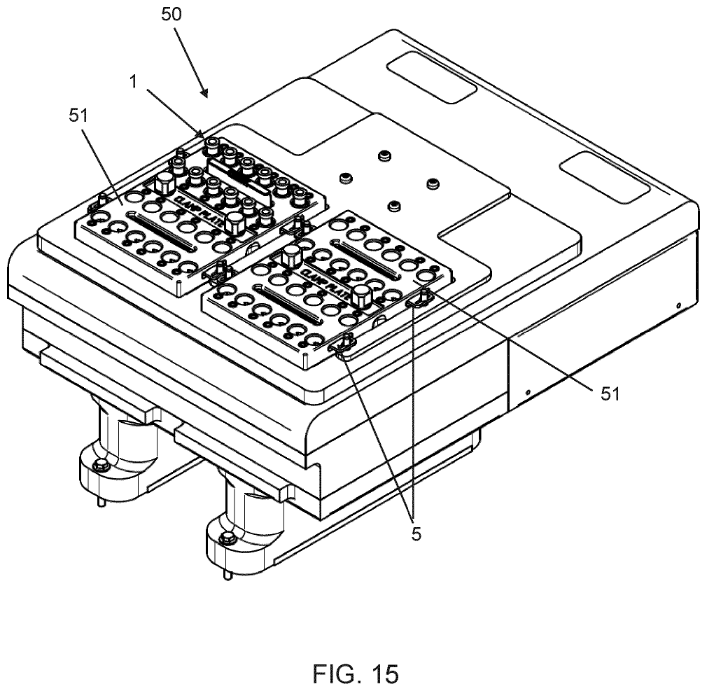

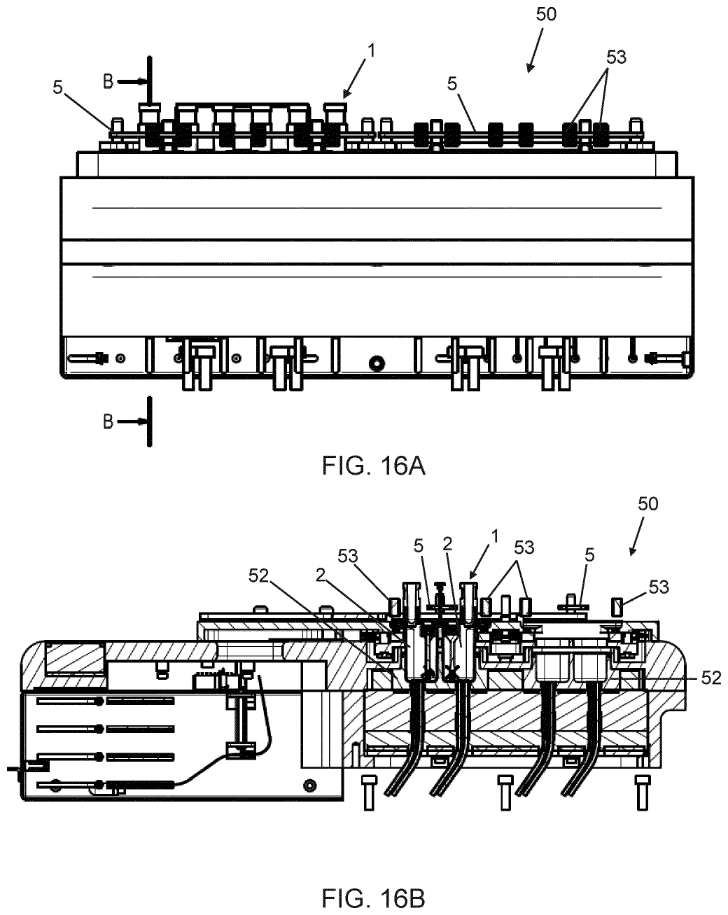

FIG. 15 shows a perspective view of a bioreactor system 50 loaded with an example of a third embodiment of a multi-parallel, single-use consumable 1, and having space for three more examples of the consumable. FIGS. 16A and 16B then show respectively a side elevation view, and a sectional view along plane B-B of the bioreactor system, but with the two clamp plates 51 (discussed below) of the system omitted to better reveal other features of the system.

In contrast to the first and second embodiments, the consumable of the third embodiment does not include a stirrer bar. Instead, the stirrer bar is provided as a permanent (i.e. non-consumable) component of the bioreactor system, as discussed in more detail below. However, the consumable of third embodiment is otherwise similar to that of the second embodiment, and, in particular, the agitators of the third embodiment are impellers. Thus FIGS. 10 and 11A to 11D described above, which show the consumable of the second embodiment with its drive mechanism omitted, also represent the consumable of the third embodiment, which does not have a drive mechanism. As in the second embodiment, each impeller of the consumable of the third embodiment comprises a shaft 31 extending into its respective bioreaction vessel, and blades 30 at the bottom end of the shaft for stirring the contents of the bioreaction. The top end of each shaft terminates in an impeller connector 32 which fits loosely around sides of a respective well formed in the lid. The bioreactor system 50 comprises a control unit (not shown) for programmably controlling the synchronised rotation of the impellers by controlling the circulating motion of the stirrer bars 5.

The bioreactor system 50 has stations for receiving four of the consumables, each having twelve bioreaction vessels 2. Each pair of stations is covered by a respective clamp plate 51 (shown in FIG. 15 but omitted in FIGS. 16A and 16B). To load the consumables into the system, the clamp plates are removed to reveal recesses into which the consumables are lowered. At the bottom of the recesses are heating blocks 52 used to control the temperature of bioreaction liquid contained within lower regions the vessels. At the top of the recesses are cooling blocks 56 used to promote condensation of off-gases in the gas head space which forms in upper regions of the vessels. The positions of the heating blocks and cooling blocks relative to the vessels correspond to the positions of the hot and cold channels described above in relation to FIG. 9. The heating and cooling blocks can be formed of thermally conductive material, such as aluminium alloy, and provide projections in the channels positioned for close contact with the walls of the vessels. The heating blocks may be heated by resistive heating elements. The cooling blocks may be cooled by Peltier cooling elements 57. The cooling blocks are thermally insulated from a covering top plate 58 of the system by thermal insulation 59. This insulation is positioned to limit heat transfer between the consumable in the vicinity of the vessel gas ports 10, 11 (discussed further below) located directly above the insulation and the cooling blocks, but allows heat transfer into the clamp plate 51. In particular, the Peltier cooling elements can reject heat into the clamp plates 51, which can also be formed of thermally conductive material, such as aluminium alloy. The control unit of the bioreactor system can programmably control the operational temperatures of the heating and cooling blocks.

Each clamp plate 51 carries two stirrer bars 5 for the two consumables which it covers. Each stirrer bar is housed in a respective slot in its clamp plate, the slot allowing the stirrer bar to move with a circulating motion, and the stirrer bar having pins at both ends which interface to an external drive linkage (not shown) for connecting to an external source of motive power of the bioreactor system, such as an electric motor.

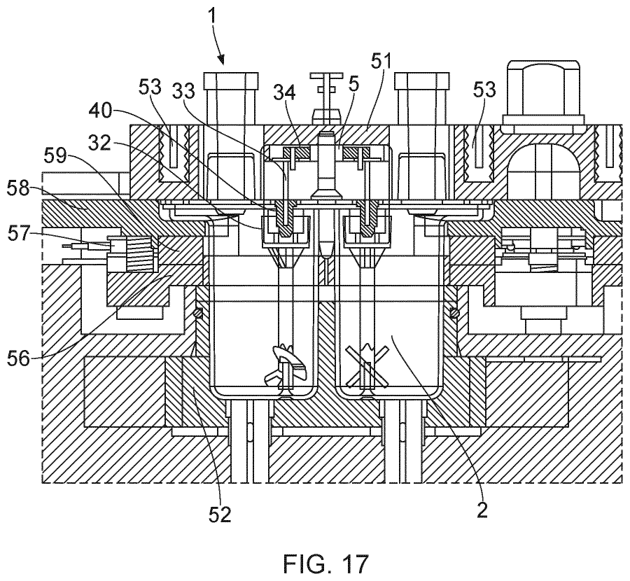

Each stirrer bar 5 also carries twelve pin wheel mechanisms of the type discussed above in relation to FIG. 13C and used to produce rotation of the impellers in the vessels. FIG. 17 is a cross-section through two neighbouring vessels of the consumable loaded in the bioreactor system 50, and shows details of the pin wheel mechanism, which comprises a pin extending from its stirrer bar 5 into a rotatable disc 34 situated in a matching recess in the clamp plate 51. When the stirrer bar is moved in a circulating motion, the rotatable discs are rotated by the pins. A drive shaft 33 connects each rotatable disc to the impeller connector 32 of the respective impeller respective via an interfacing connector 40. Thus, the circulating motion of the stirrer bar, relative to the clamp plate, drives the rotatable discs, which in-turn rotates the drive shafts to turn the impellers. The interfacing connectors 40, as discussed above in relation to FIGS. 14A and 14, self-align to the impeller connectors 32 when the clamp plates are lowered and clamped onto the consumables loaded into the stations in the bioreactor system.

Each vessel 2 of the consumable 1 of the third embodiment has an inlet gas port 11 for introducing gases to the vessel and an outlet gas port 10 through which off-gas is expelled from the vessel. These ports are at the positions shown in FIG. 10. The clamp plate 51 contains exhaust passages which form sealed gas connections to the outlet ports when the plate is lowered and clamped into place. Thus the exhaust passages can be used to remove exhaust gas from the vessels. However, unlike the vessel shown in detail in FIG. 8B, there are no tortuous exhaust channels in the lid of the vessels leading to the outlet gas ports. Rather tortuous gas paths for controlling the rate of gas exhaust are formed in the exhaust passages of the clamp plates. Conveniently this can be achieved by forming the exhaust passages as helical voids. In particular, screws 53 can be pressed into holes in the clamp plate 51 to produce extended helical paths of small cross-section where the screw threads engage with the plate, the exhaust gas having to follow these paths to exit the passages. As previously noted, the cooling elements can be arranged to reject heat into the clamp plates 51. This has an effect of heating the tortuous gas paths formed by the screws 53, and thus reduces a risk of off-gas condensation in these paths, which condensation can otherwise be a cause of undesirable fluid loss from the vessels.

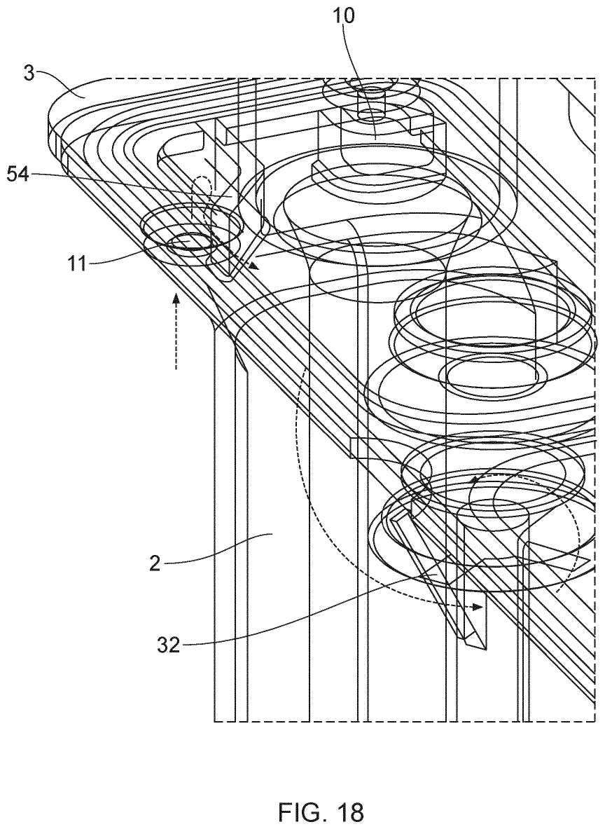

The consumable 1 of the third embodiment is configured to ensure that introduced gases mix substantially completely with gases already present in the head spaces before exiting via the outlet gas ports. In particular, as shown in the transparent perspective view of part of a vessel and lid of FIG. 18, each lid can provide a wall 54 which projects downwards from the lid and extends from between the respective inlet gas port and outlet gas port to close to the respective impeller connector 32. As shown by the arrowed lines in FIG. 18 which indicate the direction of introduced gas flow, the wall 54 guides the introduced gas into the disturbed airflow around the top of the impeller at the impeller connector, where it mixes with the existing head space gas rather than short circuiting straight to the outlet gas port. By configuring the inlet outlet gas ports in this way, substantially complete mixing of introduced gas with gas already present in the head space before it exits via the outlet gas ports can be achieved. In particular, even for small scale vessels configured to support a bioreaction working volume of between 0.5 mL and 3.5 mL, comparisons under a variety of experimental conditions show substantially no difference between (i) measured oxygen concentrations in the head space and (ii) theoretical oxygen concentrations that would be expected if there were complete mixing of introduced gas in the head space before flow to the outlet gas port.

The features disclosed in the description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

1. A consumable for a bioreactor system, the consumable comprising:
a plurality of bioreaction vessels, each suitable for holding contents of a bioreaction,
wherein the plurality of bioreaction vessels are provided as a single-piece construction, and each bioreaction vessel includes an agitator configured to agitate the contents of the respective bioreaction vessel, each agitator having a stirring end which extends into the bioreaction vessel and a drive end.

2. The consumable of claim 1, wherein the stirring ends of the agitators are paddles.

3. The consumable of claim 2, wherein the agitators are connected to a drive mechanism at their drive ends via respective ball and socket joints, or via respective living hinges.

4. The consumable of claim 2, wherein the paddles are tapered such that widths of the paddles decrease with increasing distance from their drive ends.

5. The consumable of claim 2, wherein each agitator has a pivot point for its synchronised motion between its drive end and its stirring end, and each bioreaction vessel includes a flexible element around the respective agitator at the pivot point, the flexible element providing a seal which closes off the bioreaction vessel at the agitator.

6. The consumable of claim 5, wherein the flexible element is made of a thermoplastic, elastomer or silicone.

7. The consumable of claim 1, wherein the stirring ends of the agitators are impellers.

8. The consumable of claim 1, wherein the plurality of the bioreaction vessels are rigid containers.

9. The consumable of claim 1, wherein the bioreaction vessels comprise respective outgassing ports for exhausting gas from the bioreaction.

10. The consumable of claim 9, wherein each outgassing port includes a tortuous gas path for controlled gas exhaust therethrough.

11. The consumable of claim 10, wherein the bioreaction vessels comprise respective gas inlet ports for introducing gas into the vessels.

12. The consumable of claim 11, wherein, within each bioreaction vessel, the respective outgassing port and respective gas inlet port are configured such that gas introduced through the gas inlet port mixes substantially completely with gas already present in a head space which forms in an upper region of the vessel before exiting via the outgassing port.

13. The consumable of claim 9, wherein the vessels are arranged in one or more rows, and are further arranged in side-by-side mirrored pairs along the or each of the rows.

14. The consumable of claim 1, wherein the consumable further comprises a drive mechanism connected or connectable to the drive ends of the agitators of the plurality of bioreaction vessels and configured to move the agitators with identical synchronised motions.

15. The consumable of claim 14, wherein the drive mechanism includes an interface to an external drive linkage which allows connection to an external source of motive power provided in the bioreactor system.

16. The consumable of claim 14 wherein the plurality of bioreaction vessels are arranged in one or more rows and the drive mechanism is a stirrer bar extending adjacent to and parallel with the one or more rows.

17. A bioreactor system comprising one or more of the consumables of claim 1.

18. The bioreactor system of claim 17 further comprising a source of motive power for moving the agitators of the consumable.

19. The bioreactor system of claim 18 further comprising a control unit for programmably controlling movement of the agitators.

20. A bioreactor system comprising one or more of the consumables of claim 11, the bioreactor system further comprising a non-consumable clamp plate clampable to top of the consumable and containing passages which thereby form respective sealed gas connections with the outgassing ports and/or the gas inlet ports.

21. The bioreactor system of claim 20, wherein each passage forming a sealed connection with an outgassing port includes a tortuous gas path for controlled gas exhaust therethrough.

22. The bioreactor system of claim 20, wherein the clamp plate incorporates a non-consumable drive mechanism which connects to the drive ends of the agitators of the plurality of bioreaction vessels when the clamp plate is clamped to the top of the consumable, the drive mechanism being configured to move the agitators with identical synchronised motions.

23. The bioreactor system of claim 22, wherein the plurality of bioreaction vessels are arranged in one or more rows and the drive mechanism is a stirrer bar extending adjacent to and parallel with the one or more rows.

24. The bioreactor system of claim 20, further comprising a cooling sub-system configured to promote condensation of off-gases in a gas head space which forms in upper regions of the vessels and a heating sub-system configured to perform temperature control of bioreaction liquid contained within lower regions of the vessels.

25. The bioreactor system of claim 24, wherein the cooling sub-system is arranged to reject heat from the upper regions of the vessels into the clamp plate.
